(19) United States
(12) Patent Application Publication
(10) Pub. No.: US 2014/0091494 A1
(43) Pub. Date: Apr. 3, 2014

(54) TWO-PIECE VIAL TRANSFER NEEDLE ASSEMBLY

(71) Applicant: Covidien LP, (US)

(72) Inventors: Mel Finke, Deland, FL (US); John K. Foster, Port Orange, FL (US)

(73) Assignee: Covidien LP, Mansfield, MA (US)

(21) Appl. No.: 13/847,052

(22) Filed: Mar. 19, 2013

Related U.S. Application Data
(63) Continuation of application No. 12/892,073, filed on Sep. 28, 2010, now abandoned.

(51) Int. Cl. A61M 3/00

(52) U.S. Cl. CPC A61M 3/00
USPC 264/255

(57) ABSTRACT

The present disclosure relates to a vial transfer needle assembly including a first molding member and a second molding member. The first molding member includes a hub portion that has a proximal piercing tip and a distal portion. The hub portion defines a lumen that passes therethrough. The second molding member includes a central housing, a proximal skirt, and a distal skirt. The second molding portion is molded at least partially about the first molding portion to form a monolithic structure. The piercing tip is positioned to extend into a first cavity that is defined by the proximal skirt. The distal portion of the first molding member is positioned to extend through a second cavity that is defined by the skirt. The distal skirt is dimensioned to receive a medical device.

### CROSS-REFERENCE TO RELATED APPLICATION

The present application is a continuation of U.S. patent application Ser. No. 12/892,073, filed on Sep. 28, 2010, the entire contents of which are incorporated by reference herein.

### BACKGROUND

1. Technical Field

The present disclosure relates to a vial transfer needle assembly and, more particularly, to a two-piece vial transfer needle assembly.

2. Description of Related Art

In the medical field, certain types of solutions (e.g., drugs) are commonly stored in vials that utilize a pierceable cap (e.g., resealable septum) on one end and a plunger that is inserted into the opposite end. It is common for a clinician to use a vial transfer needle assembly that includes a spike or needle on one end and a standard threaded female luer connecter on the other end to fluidly couple the vial to a medical device, e.g., syringe, catheter, hubbed needle, etc.

Currently vial transfer needle assemblies are manufactured by utilizing a three-piece design, which generally includes a plastic hub portion, a metal needle (e.g., a cannula), and a crimp-on metal insert that is used to capture the needle and seat it within the plastic hub portion. A sheath component may be pressed over the luer taper and serves as a sterile fluid path barrier when the hub is assembled to a vial.

It would be beneficial to decrease the amount of parts needed for a vial transfer needle assembly and to simplify the assembly process.

### SUMMARY

The present disclosure relates to a vial transfer needle assembly including a first molding member and a second molding member. The first molding member includes a hub portion that has a proximal piercing tip and a distal portion. The hub portion defines a lumen that passes therethrough. The second molding member includes a central housing, a proximal skirt, and a distal skirt. The second molding portion is molded at least partially about the first molding portion to form a monolithic structure. The piercing tip is positioned to extend into a first cavity that is defined by the proximal skirt. The distal portion of the first molding member is positioned to extend through a second cavity that is defined by the distal skirt. The distal skill is dimensioned to receive a medical device.

In embodiments, the vial transfer needle assembly may include a removable sheath that is configured to cover the distal portion of the first molding member. The removable sheath may be molded with the second molding member. The removable sheath may include an annular wall that defines an open proximal portion and a closed distal portion. The removable sheath is configured to cover at least a portion of the first molding member.

In other embodiments, the closed distal portion of the removable sheath may include a textured configuration to facilitate handling by a user when the removable sheath is removed therefrom.

In embodiments, the open proximal end of the removable sheath may be formed on to a portion of the distal skirt via a frangible portion such that when the removable sheath is removed the frangible portion is configured to fracture to thereby facilitate removal of the removable sheath.

The second molding member may be configured to overlay and couple to the first molding member during an injection molding process. The first molding member includes a rigid structure having a higher Shore durometer than the second molding member. Whereas, the second molding member includes a flexible material having a lower Shore durometer than the first molding member to facilitate insertion of the vial when inserted into the proximal skirt.

In embodiments, the proximal skirt includes an inner wall and a base. The inner wall may include an annular bead that is disposed along an inner periphery of the inner wall. The annular bead is provided to facilitate releasable engagement of a vial when inserted within the first cavity of the proximal skirt.

In other embodiments, the distal skirt includes an inner wall and a base. The inner wall may have a plurality of threads disposed alongside the inner wall in a helical configuration for reception of the medical device, e.g., a catheter tube, within the second cavity of the distal skirt. Connecting structures other than helical threads are envisioned.

The vial transfer needle assembly may farther include a cover that is disposed atop of an outer periphery of the proximal skirt to prevent contaminants from entering the cavity of the proximal skirt.

The present disclosure also relates to a method of manufacturing a vial transfer needle assembly. In an initial step, a first shot of a first material is injected in a form of a first molding member, the first molding member having a hub portion. Subsequently, a second shot of a second material having a lower Shore durometer than the first material is injected. The second shot is configured to at least partially overmold the first molding member. The second molding member may include a hub portion, a proximal skirt, a distal skirt, and a removable sheath.

### BRIEF DESCRIPTION OF THE DRAWINGS

Various embodiment of the subject instrument are described herein with reference to the drawings wherein:

FIG. 1 is a perspective view of a vial transfer needle assembly according to one embodiment of the present disclosure;

FIG. 2 is a side cross-sectional view of the vial transfer needle assembly of FIG. 1;

FIG. 3 is an enlarged view of the indicated area of detail of FIG. 2;

FIG. 4 is a side cross-sectional view of the vial transfer needle assembly shown in FIG. 1 with a vial having a plunger coupled thereto and a removable sheath shown removed therefrom;

FIG. 5 is an enlarged view of the indicated area of detail of FIG. 4;

FIG. 6A is a perspective view of the vial transfer needle assembly shown in FIG. 1 with a cover disposed over an opening of the vial transfer needle assembly;

FIG. 6B is a perspective view of the vial transfer needle assembly shown in FIG. 6A with the cover partially removed therefrom; and

FIG. 7 is a side cross-sectional view of the vial transfer needle assembly shown in FIG. 1 connected at one end to a vial and plunger assembly and a second end connected to a catheter tube.

### DETAILED DESCRIPTION

Embodiments of the presently disclosed vial transfer needle assembly are described in detail with reference to the drawings wherein like reference numerals identify similar or identical elements. As used herein, the term “distal” refers to that portion of the device which is further from a user while the term “proximal” refers to that portion of the device which is closer to a user.

The present invention is directed to a vial transfer needle. In order to release the solution from the vial, one end of the vial transfer needle assembly is snapped over the pierceable cap such that the spike of the vial transfer needle assembly pierces the septum of the cap. On the opposite end of the vial transfer needle assembly, a medical device is connected to the standard threaded female luer connection. The plunger in the vial is then pressed into the vial to transfer the contents of the vial through the vial transfer needle assembly and into the patient. Alternatively, the medical device, such as a syringe, connected to the threaded female luer connection may withdraw the contents from a vial through the vial transfer needle into the medical device for delivery to the patient. It is understood that the vial transfer needle of the present invention may also be used in conjunction with a plungerless vial, in which the transfer needle may also comprise self-venting features. One example of a self-venting needle is disclosed in U.S. patent application Ser. No. 12/891,885, entitled SELF-VENTING CANNULA ASSEMBLY, and filed on Sep. 28, 2010, the entirety of which is incorporated herein by reference for all purposes.

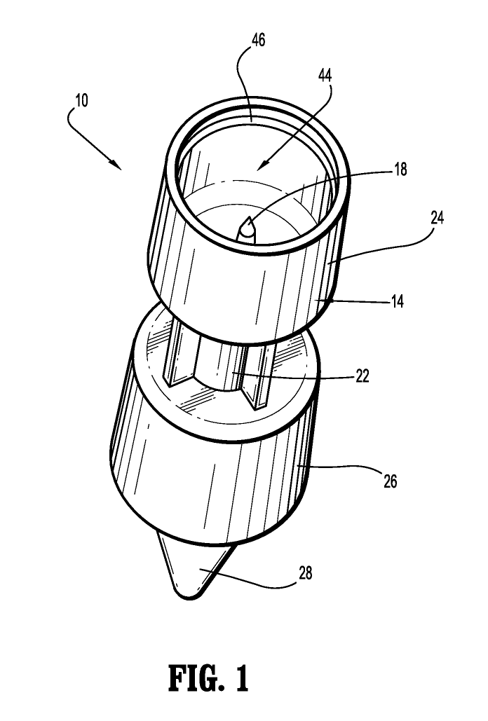

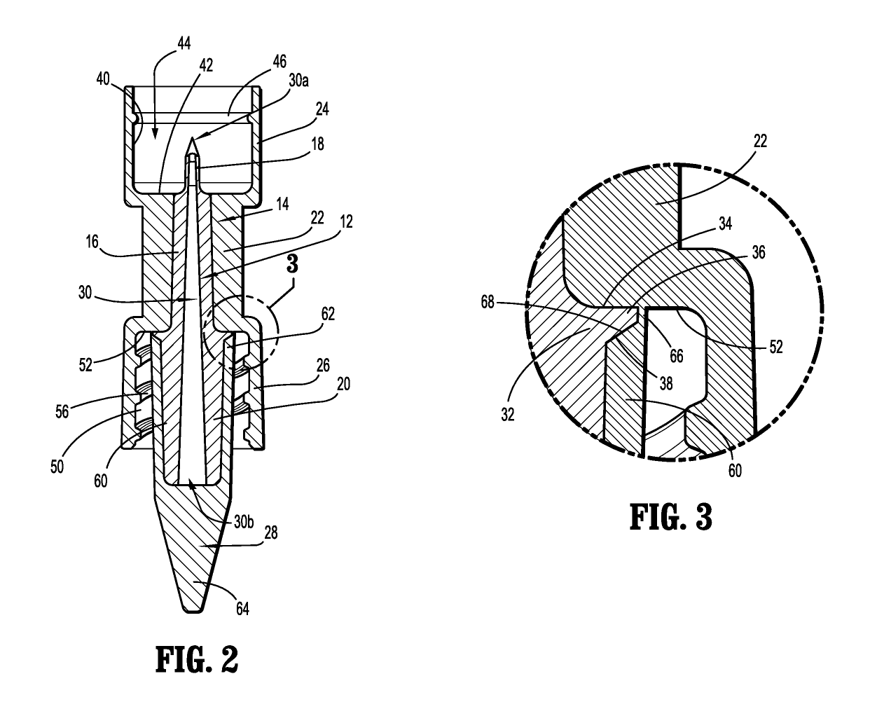

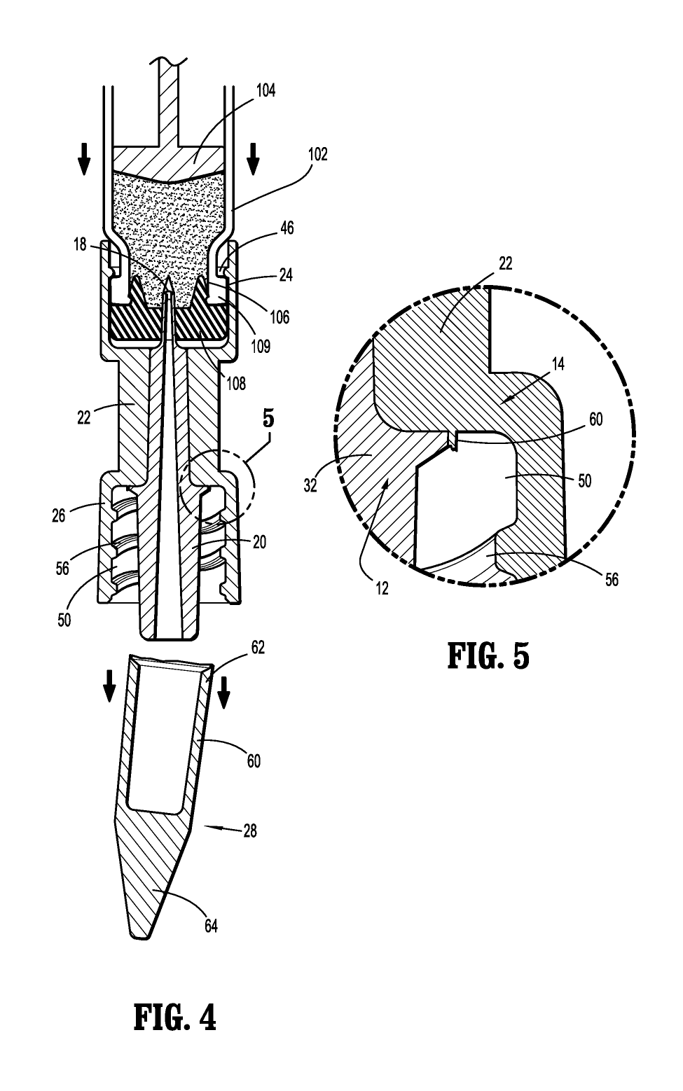

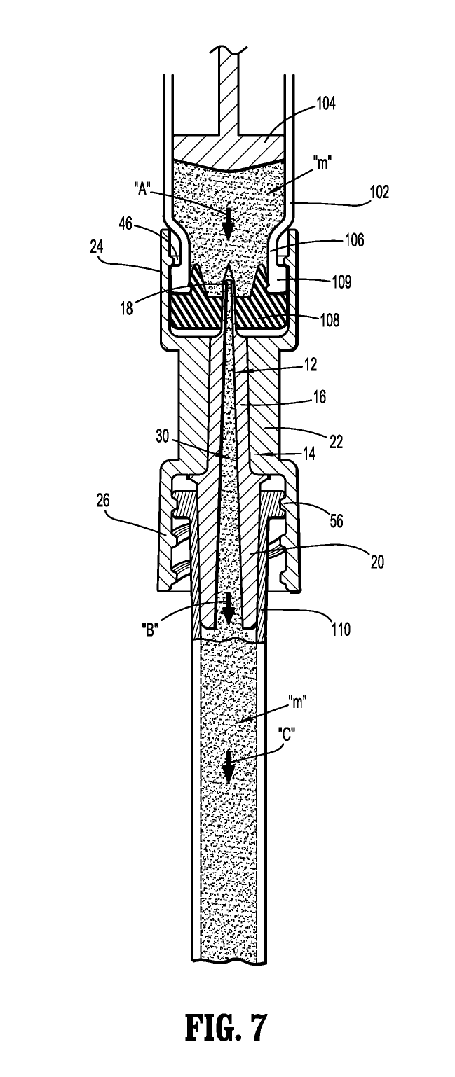

Referring initially to FIGS. 1 and 2, a transfer needle assembly is shown and generally depicted as 10. Transfer needle assembly 10 is provided to transfer fluid from a vial 102 having a plunger 104 (FIGS. 4 and 7) to a medical device. The medical device may be any medical device constructed and arranged to receive the contents of the vial and to releasably mate to the transfer needle assembly 10. For example, the medical device may be a needleless syringe or a catheter. In one embodiment shown in FIG. 7, the medical device is a catheter tube 110. Transfer needle assembly 10 generally includes a first molding member 12 and a second molding member 14 (FIG. 2) that is configured to overlay and couple to first molding member 12 during an injection molding process. In one embodiment, first molding member 12 is injected into a mold during a “first shot” of the injection molding process and, subsequently, second molding member 14 is injected over at least a portion of the first molding member 12 during a “second shot” of the injection molding process. In one embodiment, the first molding member 12 is entirely overmolded with second molding member 14. The two-shot molding process provides a benefit since it eliminates handling and costs associated with additional pieces and manufacturing steps.

Referring now to FIG. 2, first molding member 12 generally includes a central hub portion 16 having a proximal piercing tip 18 and a distal luer taper 20. Proximal piercing tip 18 is configured to pierce a pierceable septum of vial 102 (FIGS. 4 and 7). Proximal piercing tip 18 may comprise any desired shape able to pierce the septum of the vial and to transfer the contents of the vial. The piercing tip 18 may be a blunt or sharp tipped. In one embodiment, the piercing tip 18 is constructed and arranged to minimize or prevent coring of the septum during piercing.

Distal luer taper 20 is configured to be received within the medical device such as a catheter tube 110 (shown in FIG. 7). In embodiments, first molding member 12 is made from a hard material (e.g., a polymer) to provide a rigid structure for the proximal piercing tip 18 and the distal luer taper 20. In embodiments, different colors may be used to distinguish first molding member 12 from second molding member 14. For example, first molding member 12 may include one colorant, while second molding member 14 may include a different colorant. In one embodiment, first molding member 12 having a particular piercing tip configuration may be associated with one color, while a first molding member having a different piercing tip configuration may be associated with a different color. In another embodiment, different colorants may be used to identify particular assembly features, such as gage and flow.

Referring also to FIG. 2, central hub portion 16 defines a lumen 30 having proximal portion 30a and a distal portion 30b. Proximal portion 30a of lumen 30 extends to proximal piercing tip 18 and distal portion 30b of lumen 30 extends through distal luer taper 20. In embodiments, lumen 30 tapers from a larger diameter at distal portion 30b to a smaller diameter at proximal portion 30a (FIG. 2).

Central hub portion 16 also includes a shoulder 32 extending about its outer periphery to securely abut second molding member 14. Shoulder 32 includes a flat surface 34 (FIG. 3), a side surface 36 and an angled surface 38.

Second molding member 14 includes a central hub portion 22, a proximal skirt 24, a distal skirt 26 and a removable sheath 28. Proximal skirt 24 includes an inner wall 40 and a base 42 that define a cavity 44. Inner wall 40 includes an annular bead 46 that is disposed along an inner periphery of inner wall 40. Annular bead 46 is provided to facilitate releasable engagement of vial 102 within cavity 44 when the vial 102 is inserted into cavity 44 of proximal skirt 24. Other types of releasable engagement structures are known and envisioned for use in place of the annular bead. In embodiments, second molding member 14 is made from a softer material, for example, a polymer having a lower Shore durometer than first molding member 12, to allow for flexible manipulation of second molding member 14 during surgical use. For example, softer material enables proximal skirt 24 to expand when vial 102 is inserted therewithin.

Distal skirt 26 includes an inner wall 50 and a base 52 that together define a cavity 54. Inner wall 50 includes a plurality of threads 56 that are disposed alongside inner wall 50 in a helical fashion such that a catheter tube 110 (FIG. 7) or any other type of luer-type connecting conduit may be connected thereto. Connecting structure other than threads 56 is envisioned.

As discussed above, second molding member 14 includes a removable sheath 23 that is disposed within distal skirt 26. Removable sheath 28 includes an annular wall 60 defining an open proximal portion 62 and a closed distal portion 64. Removable sheath 28 is configured to cover distal luer taper 20 of first molding member 12. In embodiments, closed distal portion 64 of removable sheath 28 may have a textured, and/or ergonomic configuration to facilitate handling by a user when being pulled and/or broken off second molding member 14.

Referring to FIGS. 2 and 3, during the “second shot” of the injection molding process, open proximal end 62 of removable sheath 28 is releasably secured to base 52 of distal skirt 26 via a frangible portion 66 (e.g., very thin layer). Removable sheath 28 covers the open end of distal luer taper 20. As briefly discussed above, central hub portion 16 includes a shoulder 32, which abuts base 52 of distal skirt 26 to provide a firm placement of central hub portion 16 in relation to second molding member 14. In addition, during the “second shot” of the injection molding process, the softer material is configured to take a form over shoulder 32. More specifically, an angled surface 68 of proximal portion 62 of removable sheath 28 abuts angled surface 38 of shoulder 32, while frangible portion 66 surrounds side surface 36 of shoulder 32. Referring also to FIGS. 4 and 5, frangible portion 66 is configured to easily break to facilitate removal of removable sheath 22 from second molding member 14 prior to use of vial transfer needle assembly 10.

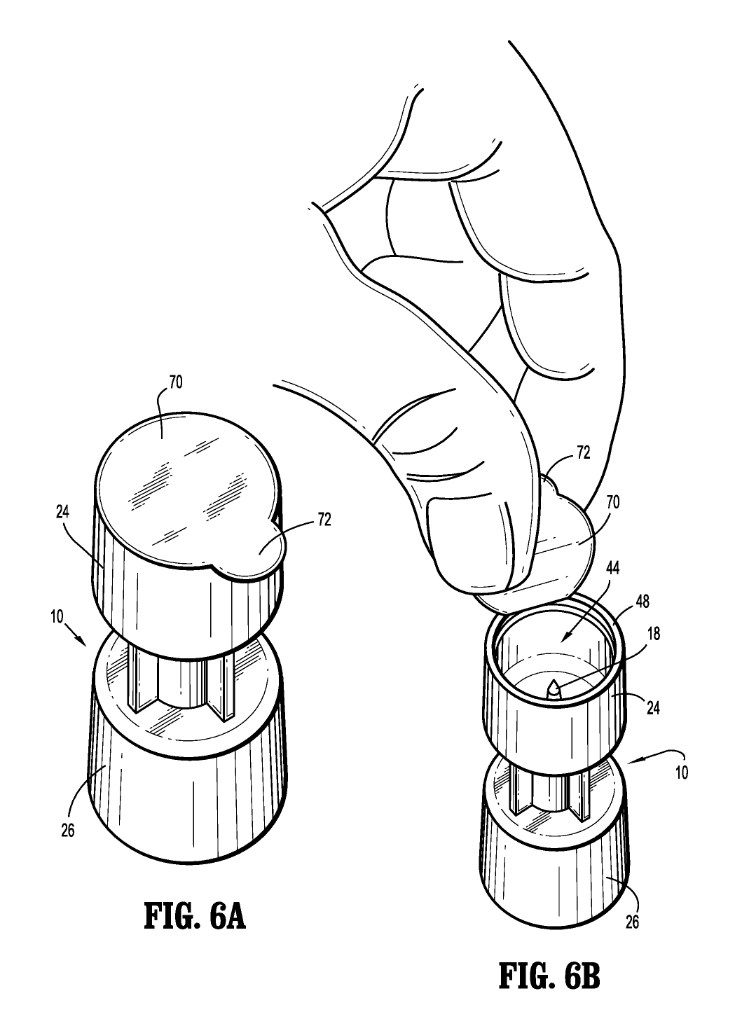

Referring to FIGS. 6A and 6B, a cover 70 may be disposed over the opening defined by an outer periphery 48 of proximal skirt 24. Cover 70 may have a pull tab 72 and is configured to prevent any contaminants from entering cavity 44 of proximal skirt 24 during packaging and before a surgical procedure is performed. Cover 70 may be formed of any conventional material such as a coated paper, laminate or heat sealable film.

Referring to FIG. 7, as it is commonly known in the art, vial 102 may contain a medicinal fluid “M” or any other suitable types of fluids. As it is also commonly known in the art, vial 102 may include a plunger 104, a neck portion 106, and a pierceable septum 108 that is positioned within a rim 109 of vial 102.

During use, a clinician removes (e.g., peels off) cover 70 from outer periphery 48 of proximal skirt 24 and inserts vial 102 within cavity 44 of proximal skirt 24. As vial 102 is inserted within cavity 44, rim 109 of vial 102 is pushed passed annular bead 46 formed on an inner wall of proximal skirt 24 until septum 108 abuts base 42 of the proximal skirt 24. In this position, annular bead 46 is securely snug around neck portion 106, thus securing vial 102 within proximal skirt 24. Also in this position, proximal piercing tip 18 pierces the pierceable septum 108 of vial 102 to thereby allow medicinal fluid “M” to pass through proximal lumen 30a.

The clinician may then remove removable sheath 28 by twisting and/or pulling on removable sheath 28, thus breaking the frangible portion 66. After removing the removable sheath 28, the clinician then inserts a connecting structure, e.g., a liter connector, of the cannula tube 110 or other medical device, e.g., syringe, catheter, blabbed needle, etc., into distal skirt 26 into engagement with luer threads 56 such that distal luer taper 20 is disposed within cannula tube 110. Afterwards, plunger 104 of vial 102 is depressed such that the medicinal fluid “M” travels from vial 102 (as shown by arrow “A”), through central hub portion 16 and distal liter taper 20 (as shown by arrow “B”) via lumen 30, and ultimately into catheter tube 110 (as shown by arrow “C”).

It will be understood that various modifications may be made to the embodiments disclose herein. For example, the disclosed removable sheath may be provided after the disclosed second shot molding process. That is, the disclosed removable sheath may be made from a different material other than a polymer, such as, for example, any suitable type of metal. Therefore, the above description should not be construed as limiting, but merely as exemplifications of embodiments. Those skilled in the art will envision other modification within the scope and spirit of the claims appended hereto.

What is claimed:

1. A two shot process for injection molding a vial transfer needle assembly, comprising:
injecting a first molding material into a mold during a first shot of the injection molding process to form a first molding member, the first molding member including a hub portion defining a lumen; and
injecting a second molding material into the mold over the first molding member during a second shot of the injection molding process to overlay and couple the second molding member to the first molding member, the second molding member including a central housing, a proximal skirt, and a distal skirt.

2. The method of claim 1, wherein the step of injecting the first molding material includes forming the first molding member such that the first molding member has a proximal piercing tip and a distal portion, the lumen extending from the proximal piercing tip to the distal portion.

3. The method of claim 2, wherein the step of injecting the second molding material includes forming the second molding member such that the piercing tip is positioned to extend into a first cavity defined by the proximal skirt and the distal portion of the first molding member is positioned to extend through a second cavity defined by the distal skirt.

4. The method of claim 1, wherein the first molding member is formed to have a higher Shore durometer than the second molding member.

5. The method of claim 1, wherein the second material is different from the first material.

6. The method of claim 1, wherein the step of injecting the second molding material includes completely overmolding the second molding member over the first molding member.

7. The method of claim 6, wherein the step of injecting the second molding material includes completely overlying the second molding member about the first molding member such that the first molding member and the second molding member are integrally formed.

8. The method of claim 1, wherein the step of injecting the first molding material includes forming the first molding member such that the lumen of the hub portion has a tapered configuration.
